(19) United States
(12) Patent Application Publication
(10) Pub. No.: US 2015/0322468 A1
(43) Pub. Date: Nov. 12, 2015

(54) METHOD OF PRODUCING BIOFUEL USING MICROALGAE CULTURES

(71) Applicant: Arizona Board of Regents on behalf of Arizona State University, Scottsdale, AZ (US)

(72) Inventor: Qiang Hu, Chandler, AZ (US)

(21) Appl. No.: 14/705,837

(22) Filed: May 6, 2015

Related U.S. Application Data
(60) Provisional application No. 61/989,961, filed on May 7, 2014.

(51) Int. Cl. C12P 7/64; C12N 15/113

(52) U.S. Cl. CPC C12P 7/6463; C12N 15/1137; C12N 2310/14

(57) ABSTRACT

The present invention relates to methods of improving TAG production of microalgae, for example, the microalgae in microalgal mass culture systems, and compositions of the improved microalgae. The method of improving TAG production may be increasing the yield of TAG produced by each microalgae or increasing the total yield of a mass culture system by increasing the number of microalgae in the mass culture system.

### CROSS REFERENCE TO RELATED APPLICATIONS

This document claims the benefit of U.S. Provisional Patent Application 61/989,961, filed on May 7, 2014, the contents of which are hereby incorporated by reference thereto for all purposes in its entirety.

### BACKGROUND OF THE INVENTION

Under optimal conditions of growth, microalgae synthesize fatty acids principally for esterification into glycerol-based membrane lipids, which constitute about 5-20% of their dry cell weight (DCW). Fatty acids include medium-chain (C10-C14), long-chain (C16-18) and very-long-chain (≧C20) species and fatty acid derivatives. The major membrane lipids are the glycosylglycerides (e.g. monogalactosyldiacylglycerol, digalactosyldiacylglycerol and sulfoquinovosyldiacylglycerol), which are enriched in the chloroplast, together with significant amounts of phosphoglycerides (e.g. phosphatidylethanolamine (PE) and phosphatidylglycerol (PG)), which mainly reside in the plasma membrane and many endoplasmic membrane systems (Guckert and Cooksey, 1990; Harwood, 1998; Pohl and Zurheide, 1979a,b; Wada and Murata, 1998). The major constituents of the membrane glycerolipids are various kinds of fatty acids that are polyunsaturated and derived through aerobic desaturation and chain elongation from the ‘precursor’ fatty acids palmitic (16:0) and oleic (18:1×9) acids (Erwin, 1973).

Under unfavorable environmental or stress conditions for growth, however, many microalgae alter their lipid biosynthetic pathways towards the formation and accumulation of neutral lipids (20-50% DCW), mainly in the form of triacylglycerol (TAG). Unlike the glycerolipids found in membranes, TAGs do not perform a structural role but instead serve primarily as a storage form of carbon and energy. However, there is some evidence suggesting that, in microalgae, the TAG biosynthesis pathway may play a more active role in the stress response, in addition to functioning as carbon and energy storage under environmental stress conditions. Unlike higher plants where individual classes of lipid may be synthesized and localized in a specific cell, tissue or organ, many of these different types of lipids occur in a single algal cell. After being synthesized, TAGs are deposited in densely packed lipid bodies located in the cytoplasm of the algal cell, although formation and accumulation of lipid bodies also occur in the inter-thylakoid space of the chloroplast in certain green microalgae, such as Dunaliella bardawil (Ben-Amotz et al., 1989). In the latter case, the chloroplastic lipid bodies are referred to as plastoglobuli.

Microalgae have been considered as an alternative feedstock for advanced liquid fuels such as biodiesel and bio jet fuel due to their ability to produce substantial amounts of TAG, rapid growth potential, and tolerance to environmental conditions (Hu et al., 2008; Sheehan et al., 1998). Production of TAG from current microalgal mass culture systems has been considerably lower in reality compared to the theoretical maximum (Hu et al., 2008; Sheehan et al., 1998). Thus there is a need for methods of improving the TAG production from micoalgal mass culture systems.

### SUMMARY OF THE INVENTION

The invention is directed to methods of improving TAG production of microalgae, for example, the microalgae in microalgal mass culture systems, and compositions of the improved microalgae.

In one embodiment, the methods of improving TAG production of microalgae comprises reducing the activity of AGPase in microalgae. The method may further comprise culturing the microalgae in nitrogen-deplete growth conditions. In some aspects, the method further comprises expressing a particular PDAT with broad substrate specificity.

In another embodiment, the methods of improving TAG production of microalgae comprises expressing a particular PDAT with broad substrate specificity. The methods may further comprise reducing the activity of AGPase in microalgae. As such, in some aspects culturing the microalgae in nitrogen-deplete growth conditions.

In still another embodiment, the method of improving TAG production of microalgae comprising reducing the activity of GPAT in microalgae. RNAi technology may be used to reduce the activity of GPAT, for example, through administration of GPAT siRNA. In some aspects, the methods comprise culturing the microalgae in nitrogen-replete conditions. In other aspects, the methods comprise culturing the microalgae in nitrogen-deplete conditions.

### BRIEF DESCRIPTION OF THE FIGURES

FIG. 1 depicts the pathways for production of TAG in microalgae.

FIG. 2 depicts metabolism of 3-phosphoglyceric acid (3-PGA) for starch synthesis and lipid synthesis.

FIG. 3 depicts a pathway for TAG synthesis that is independent of acyl-CoA (coenzyme A) in microalgae. The pathway is mediated by phospholipid:diacylglycerol acyltransferase (PDAT) that uses phospholipids as an acyl donors and diacylglycerol (DAG) as an acyl accepter to produce TAG. PDAT can use glycolipid, for example monogalactolipid, as acyl donors to produce TAG.

FIG. 4 depicts the acyl-CoA-dependent pathway for TAG synthesis in microalgae, which is also known as the Kennedy Pathway.

FIG. 5 depicts the spatial localization of lipid bodies in the CW15 and Sta6 strains of Chlamydomonas reinhartdtii in nitrogen-replete (N+) and nitrogen-deplete (N−) conditions. The darker gray areas are indicative of the size and morphology of the chloroplast while the lighter gray areas depict the lipid droplets. It is apparent that lipid droplets are associated with the chloroplast.

FIG. 6 depicts the electron microscopy image of the intimate association of lipid droplets with the envelope membranes of the chloroplast.

FIG. 7 depicts the hypothesis that the formation of lipid droplets in microalgae occurs to certain sub-domains of the chloroplast where TAG are synthesized and accumulated between the two leaflets of the envelope membranes of the chloroplast. When sufficient amounts of TAG are accumulated, oil droplet-like organelles form.

FIG. 8 depicts the hyper-accumulation (9-fold increase) of oil/oil droplets in the ADP-glucose pyrophosphorylase (AGPase, NCBI ID: AF193431, EMBL UniProt ID: Q9LLL6) mutant (lower panel), as compared to the wild type (upper panel).

FIG. 9 depicts that glycerol-3-phosphate acetyrtransferase (GPAT, EMBL UniProt ID: H9CTH0, JGI v5.5 ID: Cre06.g273250) is responsible for TAG synthesis (A) and is targeted to the endoplasmic reticulum and the mitochondria (B). More GPAT are expressed in mitochondria than in endoplasmic reticulum under stress, which corresponds to accumulation of lipid droplets (C).

FIG. 10 depicts that the knock down of GPAT expression (A) also resulted in increased cell reproduction as measured by cell count and dry weight (B). The reduction of GPAT activity also changes in the synthesis of TAG and chloroplast membrane lipids (glycolipid and phospholipid) in nitrogen-replete (C) and nitrogen-deplete (D) growth conditions. MGDG=monogalactosyldiacylglycerol, DGDG=digalactosyldiacylglycerol; PG=phosphatidyl-glycerol; PI=phosphatidylinositol; and SQDG=sulfoquinovosyldiacylglycerol (SQDG).

FIG. 11 depicts the characterization of a PDAT (EMBL UniProt ID: H6V961, JGI v5.5 ID: Cre02.g106400) with multiple lipase function in addition to the known hydrolase function. The particular PDAT can convert neutral lipids, phospholipids and glycolipids, into free fatty acids or fatty acid methyl esters (or biodiesel) with 80-95% efficiency in addition to the conversion of TAG.

### DETAILED DESCRIPTION OF THE INVENTION

The verb “comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one.”

As used herein, the term “microalgae” refers to unicellular algae. Examples of microalgae include Botryococcus braunii, C. reinhardtii, Chlorella species, Dunaliella tertiolecta, Dunaliella bardawil, Gracilaria species, Pleurochrysis carterae, and Sargassum species.

As used herein, the terms “oil” and “lipid” may be used interchangeably to refer to fatty acids synthesized by microalgae.

The invention is directed to method of improving biofuel production, for example TAG production, of microalgae used in microalgal mass culture systems by metabolic engineering of selected microalgae to increase production of TAG or polyunsaturated fatty acids by each organism and/or by increasing the amount of microalgae in the mass culture to result in a greater total production. The invention is also directed to compositions and microalgal mass culture systems comprising the improved microalgae.

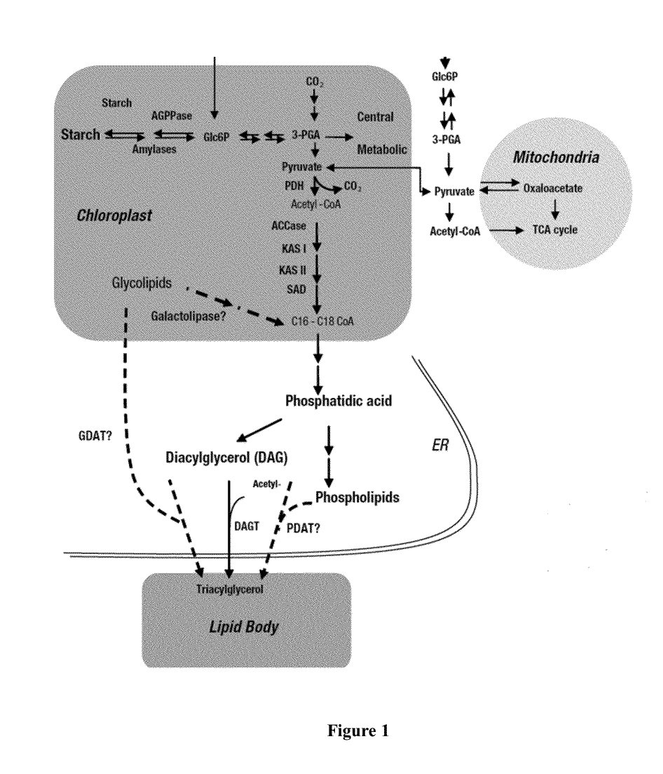

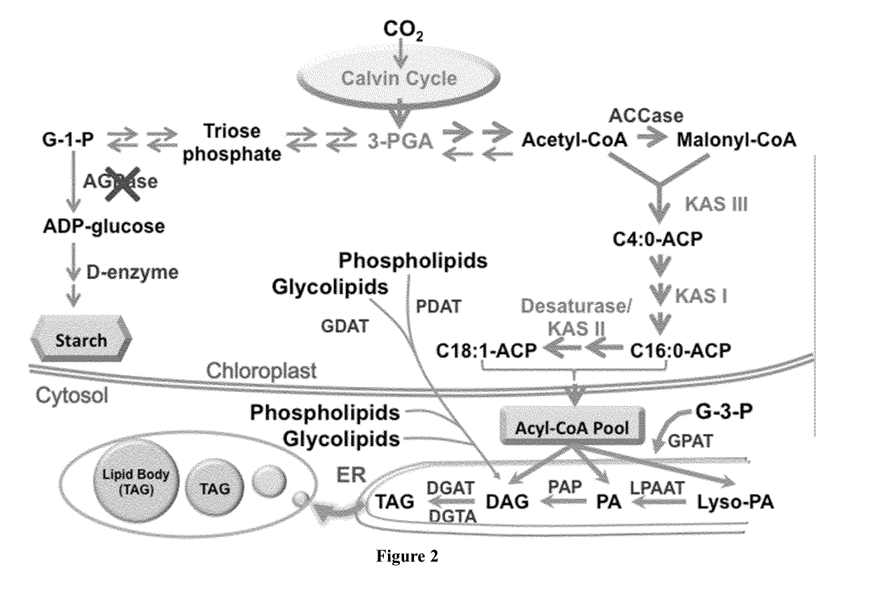

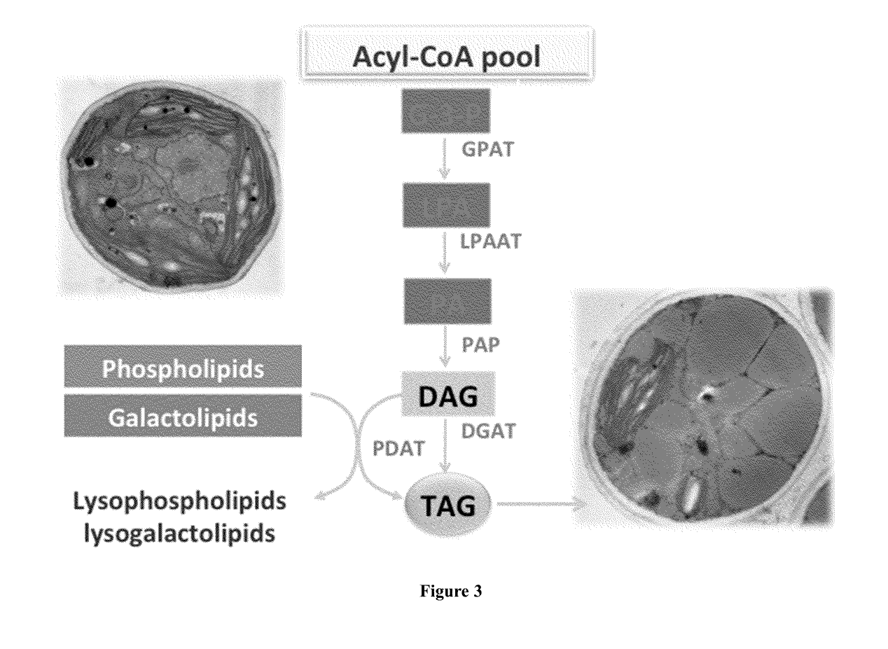

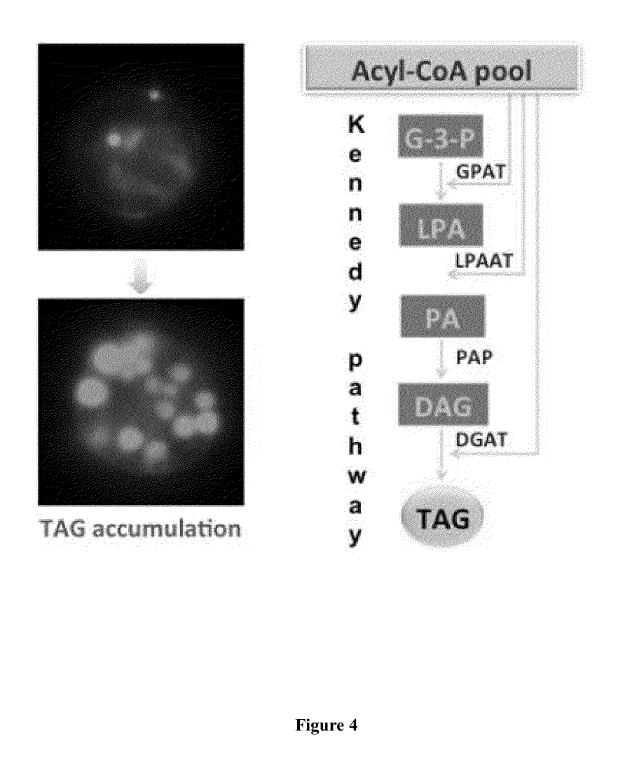

In one aspect, TAG production is increased by altering the metabolism of the microalgae to produce more TAG per gram of dry weight or per cell. Starch synthesis and lipid synthesis shares common carbon precursors in microalgae (FIGS. 1 and 2). Thus in one implementation, microalgae cells may be modified to have reduced starch synthesis activity, for example, through the reduction AGPase activity. The agents for reducing AGPase activity may be inhibitors of AGPAse protein expression or gene expression (e.g. RNAi technology such as siRNA) or through direct inhibitors of AGPase activity that act on the enzyme itself or its activated effector proteins. The inactivation of AGPase shunts photosynthetic carbon precursors to lipid synthesis to result in greater TAG production. In some embodiments, TAG production may be further enhanced by culturing microalgae with reduced starch synthesis activity in unfavorable, for example nitrogen-deplete, growth conditions. As depicted in Example 1, the Sta6 strain of C. reinhardtii, which has faulty starch synthesis, has more rapid lipid production in nitrogen-deplete growth conditions compared to the strain grown in nitrogen-replete conditions and the CW15 strain having no alterations in the starch synthesis pathway.

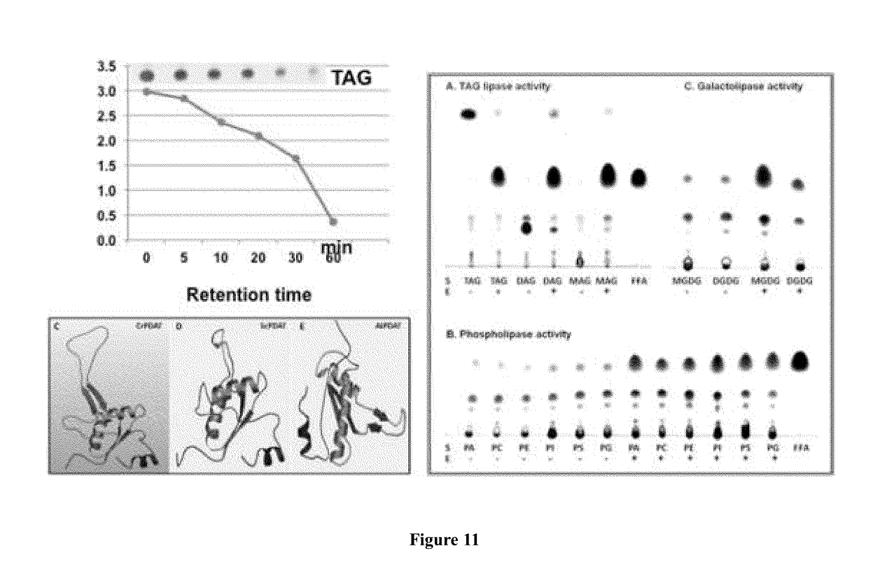

In another implementation of altering the metabolism of the microalgae in the mass culture to produce more TAG per gram of dry weight or per cell, microalgae cells may be modified to express a particular PDAT with broad substrate specificity. It is well understood in the art how to express exogenous genes in microalgae, for example, by genetic transformation. The particular PDAT has the ability to convert neutral lipids such phospholipids and glycolipids into fatty acids or fatty acid methyl esters (FIG. 11). As the particular PDAT may breakdown more varieties of starting material, more TAG can be produced by the microalgae expressing the particular PDAT compared to a microalgae expressing regular PDAT.

As PDAT and AGPase are enzymes of different pathways, TAG production may be improved microalgae by reducing starch synthesis and having the microalgae express PDAT with broad substrate specificity. Accordingly, in one embodiment of the methods for improving biofuel production of microalgae, the microalgae may be modified to have reduced AGPase activity as well as modified to express the particular PDAT.

In another aspect, TAG production is increased through enhanced cell reproduction by increasing the number of microalgal cells in the mass culture system. In one implementation, microalgae may be modified to have reduced GPAT activity. The agents for reducing GPAT activity may be inhibitors of GPAT protein expression or gene expression (e.g. RNAi technology such as siRNA) or direct inhibitors of GPAT activity that act on the enzyme itself or its activated effector proteins. As demonstrated in Example 2, knock-down of GPAT increased the growth of microalgae as measured by cell count and dry weight in both nitrogen-replete and nitrogen-deplete growth conditions. Thus in one embodiment, the microalgae may be improved for TAG production by knocking down the expression of GPAT with siRNA.

In some aspects, the invention is directed to temporary modifications of microalgae. For example, the microalgae may be first modified for enhanced cell reproduction by reducing their GPAT activity. After the agents for reducing GPAT activity no longer acts on GPAT, for example, when the agent has been removed by degradation or exocytosis, the microalgae may be modified to increase the production of TAG per microalgal cell, such as by inducing the expression of the particular PDAT and/or reducing AGPase activity.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

1. Knock-down of AGPase increases 9-fold increase in TAG production in microalgae

C. reinhardtii with mutations in the STA6 locus resulting in the failure to accumulate starch and the lack of ADP-glucose pyrophosphorylase activity was used to determine the impact of inhibiting starch synthesis on TAG production. The CW15 strain, which only has defects in the cell wall, was used to represent the amount TAG production of C. reinhardtii having normal starch synthesis pathways.

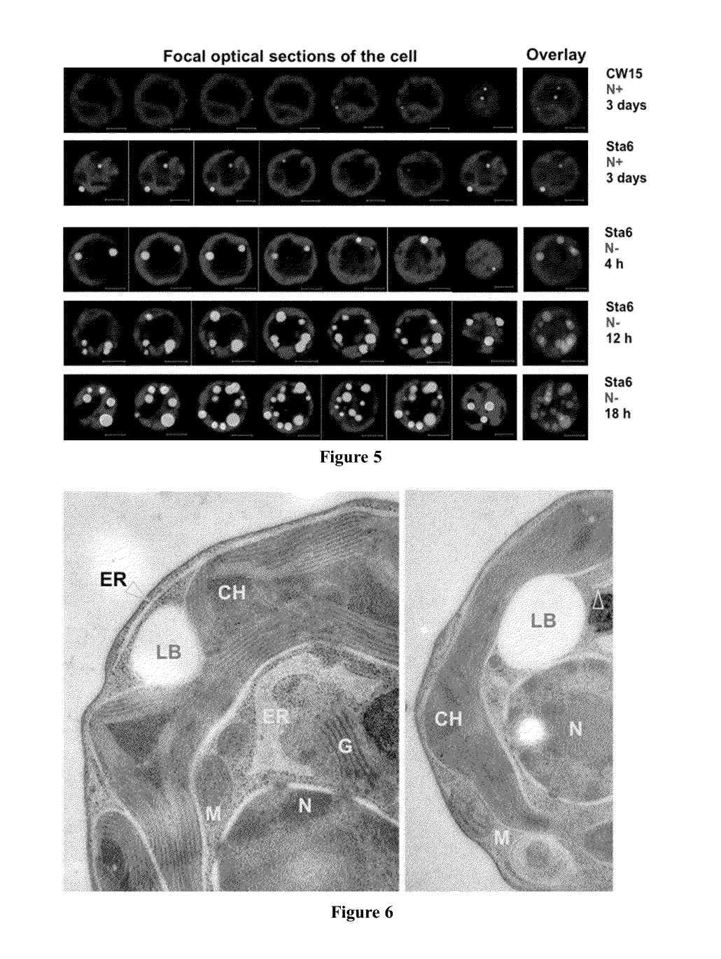

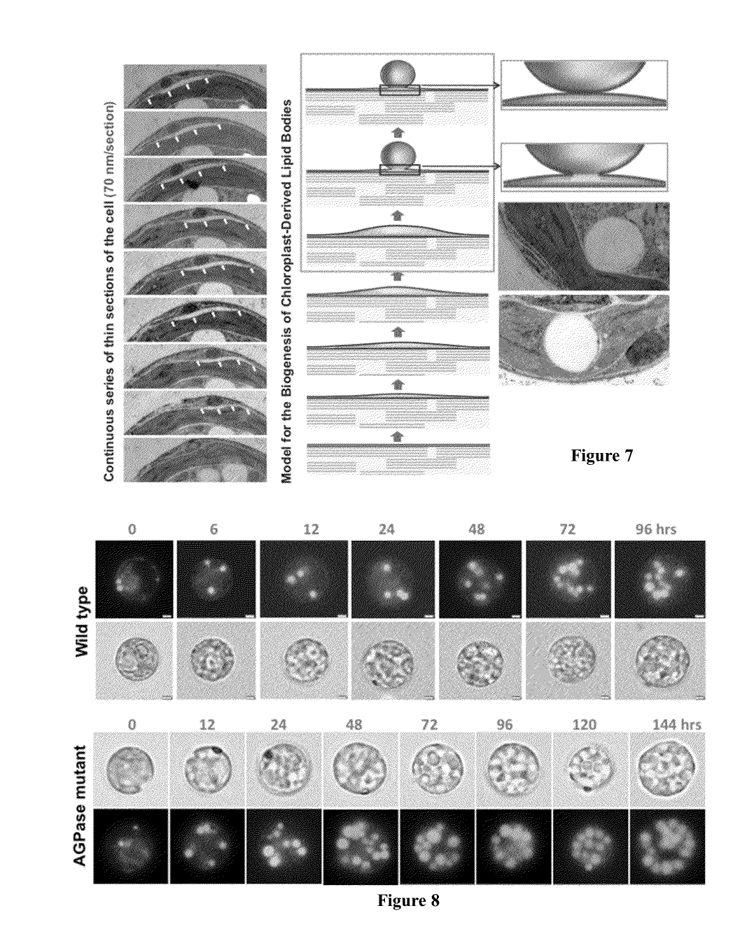

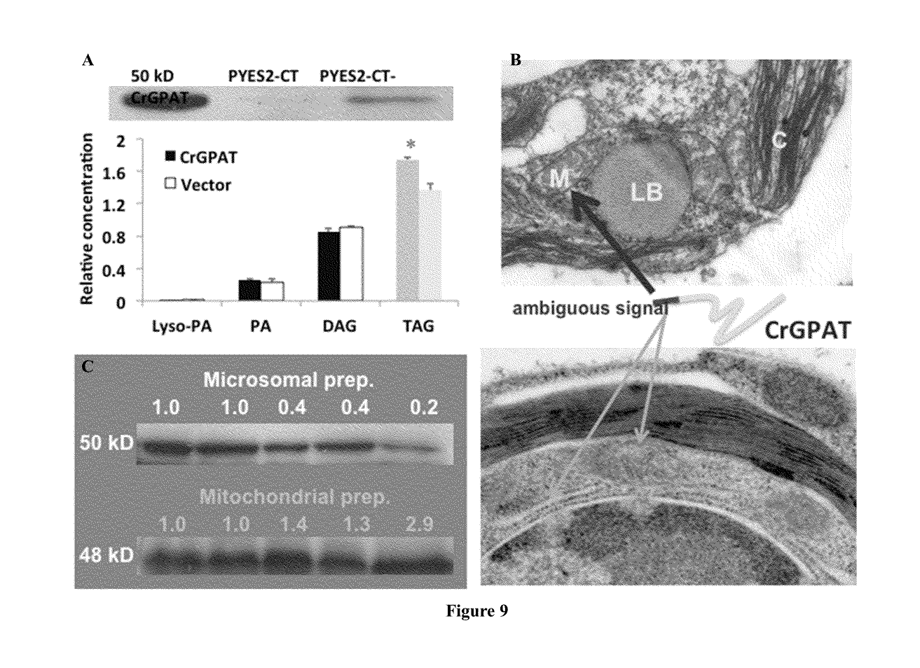

When both CW15 and Sta6 strains of C. reinhardtii were cultured in nitrogen-replete conditions for three days, the Sta6 strain produced more visible lipid droplets (FIG. 5). The increase in TAG production of Sta6 strain is more pronounced in nitrogen-deplete conditions. After fours hours of culturing in nitrogen-deplete conditions, the amount and size of lipid droplets visible was increased significantly compared to that of the strain grown in nitrogen-replete conditions. The amount and size of the lipid droplets in the Sta6 strain also increased significantly between four hours of culturing in nitrogen-deplete conditions and 12 hours of culturing in nitrogen-deplete conditions and even more significantly after 18 hours of culturing in nitrogen-deplete conditions.

2. Knock-down of GPAT increases growth potential of microalgae

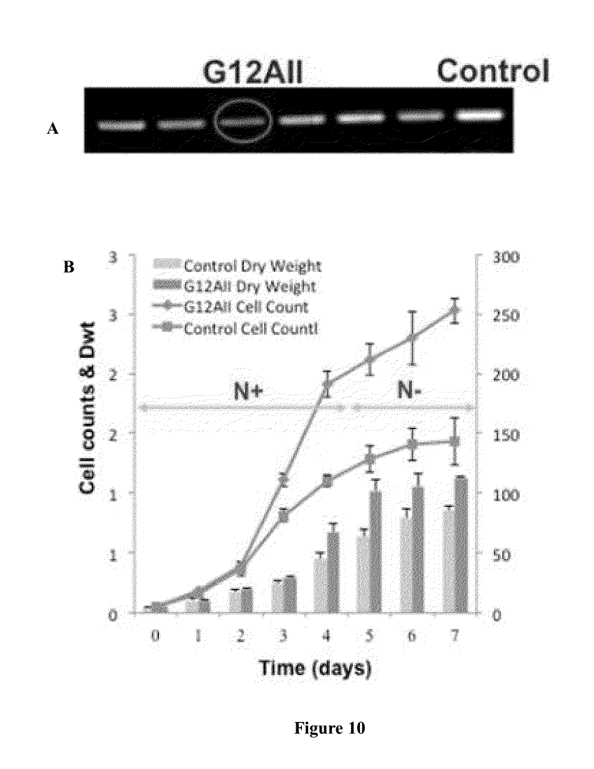

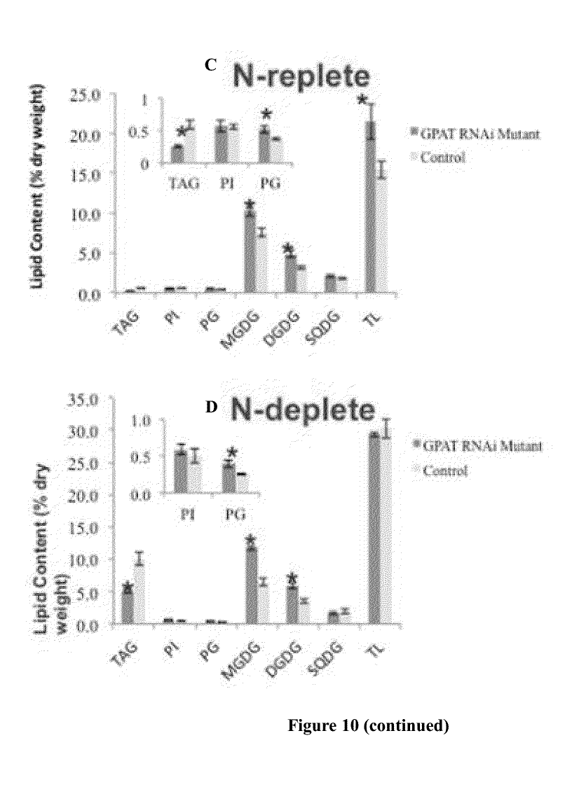

GPAT was knocked down using RNAi technologies to produce a strain of C. reinhardtii named G12AII. When cultured in nitrogen-replete conditions for the first four days, the number of G12AII cells was significantly more than the number of control cells by day 3. The difference was further increased by day 4 (FIG. 10B). At day 5, the culturing conditions were switched to nitrogen-deplete conditions. Whereas the cell number of control cells did not changed significantly through during the nitrogen-deplete growth conditions, the cell number of G12AII cells were significantly difference between day 5 and day 7 (FIG. 10B). Thus even in nitrogen-deplete conditions, there was more cell reproduction in G12AII than control cells.

The dry weight of the cell cultures was significantly different between the control cells and G12AII cells by day 4 and maintained significant for the rest of the study period (FIG. 10B). In contrast to the cell number, the dry weight of the each of the cell types were not significantly different through the period of nitrogen depletion. Neither the control cells nor G12AII cells had significantly different dry weight between day 5 and days 6 or 7.

During the period of nitrogen repletion, the control cells had significantly more TAG in the produced lipid content as a percentage of dry weight than G12AII while G12AII had significantly more phosphoglycerol in the produced lipid content as a percentage of dry weight (FIG. 10C). This difference was maintained during the period of nitrogen depletion (FIG. 10D). For both culturing conditions, knock down of GPAT resulted in 60-70% decrease in TAG synthesis in cytosol and enhanced synthesis of chloroplast membrane lipids (glycolipid and phospholipid) and protein synthesis. The results suggest enhanced photosynthesis and cell reproduction in G12AII cells.

### REFERENCES

Ben-Amotz, A., Shaish, A. and Avron, M. (1989) Mode of action of the massively accumulated b-carotene of Dunaliella bardawil in protecting the alga against damage by excess irradiation. Plant Physiol. 91, 1040-1043.

Erwin, J. A. (1973) Comparative biochemistry of fatty acids in eukaryotic microorganisms. In Lipids and Biomembranes of Eukaryotic Microorganisms (Erwin, J. A., ed.). New York: Academic Press, pp. 141-143.

Guckert, J. B. and Cooksey, K. E. (1990) Triacylglyceride accumulation and fatty acid profile changes in Chlorella (Chlorophyta) during high-pH induced cell cycle inhibition. J. Phycol. 26, 72-79.

Harwood, J. L. (1998) Membrane lipids in algae. In Lipids in Photosynthesis: Structure, Function and Genetics (Siegenthaler, P. A. and Murata, N., eds). Dordrecht, The Netherlands: Kluwer Academic Publishers, pp. 53-64.

Hu, Q., Sommerfeld, M., Jarvis, E., Ghirardi, M., Posewitz, M., Seibert, M., Darzins, A., 2008. Microalgal triacylglycerols as feedstocks for biofuel production: perspectives and advances. Plant J. 54, 621-639.

Pohl, P. and Zurheide, F. (1979a) Fatty acids and lipids of marine algae and the control of their biosynthesis by environmental factors. In Marine Algae in Pharmaceutical Science (Hoppe, H. A., Levring, T. and Tanaka, Y., eds). Berlin: Walter de Gruyter, pp. 473-523.

Pohl, P. and Zurheide, F. (1979b) Control of fatty acid and lipid formation in Baltic marine algae by environmental factors. In Advances in the Biochemistry and Physiology of Plant Lipids (Appelqvist, L. A. and Liljenberg, C., eds). Amsterdam: Elsevier, pp. 427-432.

Sheehan, J., Dunahay, T., Benemann, J., Roessler, P. G., 1998. US Department of Energy's Office of Fuels Development, July 1998. A Look Back at the US Department of Energy's Aquatic Species Program—Biodiesel from Algae, Close Out Report TP-580-24190. Golden, CO: National Renewable Energy Laboratory.

Wada, H. and Murata, N. (1998) Membrane lipids in cyanobacteria. In Lipids in Photosynthesis: Structure, Function and Genetics (Siegenthaler, P. A. and Murata, N., eds). Dordrecht, The Netherlands: Kluwer Academic Publishers, pp. 65-81.

What is claimed is:

1. A method of improving TAG production of microalgae comprising reducing the activity of AGPase in microalgae.

2. The method of claim 1, further comprises culturing the microalgae in nitrogen-deplete growth conditions.

3. The method of claim 1, further comprising expressing a particular PDAT with broad substrate specificity.

4. A method of improving TAG production of microalgae comprising expressing a particular PDAT with broad substrate specificity.

5. The method of claim 4, further comprising reducing the activity of AGPase in microalgae.

6. The method of claim 5, further comprises culturing the microalgae in nitrogen-deplete growth conditions.

7. A method of improving TAG production of microalgae comprising reducing the activity of GPAT in microalgae.

8. The method of claim 7, wherein reducing the activity of GPAT using RNAi technology.

9. The method of claim 8, wherein the RNAi technology is GPAT siRNA.

10. The method of claim 7, wherein the microalgae is cultured in nitrogen-replete conditions.

11. The method of claim 7, wherein the microalgae is cultured in nitrogen-deplete conditions.
